(19) United States
(12) Patent Application Publication
(10) Pub. No.: US 2013/0071773 A1
(43) Pub. Date: Mar. 21, 2013

(54) HOLOGRAPHIC STORAGE MEDIUM

(71) Applicants: Gary Davis, Albany, NY (US); Shantaram Narayan Naik, Bangalore (IN); Kiran Arunkumar Puthamane, South Kodagu (IN); George Kuriakose, Bangalore (IN); Sumeet Jain, Schenectady, NY (US); Vinodkumar Vasudevan, Alleppy (IN); Prashant Patil, Dist-Sindhudurg (IN)

(72) Inventors: Gary Davis, Albany, NY (US); Shantaram Narayan Naik, Bangalore (IN); Kiran Arunkumar Puthamane, South Kodagu (IN); George Kuriakose, Bangalore (IN); Sumeet Jain, Schenectady, NY (US); Vinodkumar Vasudevan, Alleppy (IN); Prashant Patil, Dist-Sindhudurg (IN)

(73) Assignee: SABIC INNOVATIVE PLASTICS IP B.V., Bergen op Zoom (NL)

(21) Appl. No.: 13/234,576

(22) Filed: Sep. 16, 2011

(51) Int. Cl. G03F 7/032; C07C 237/30; C07C 237/40; C07D 333/24

(52) U.S. Cl. 430/2; 549/77; 564/163; 564/168

(57) ABSTRACT

Disclosed herein are novel amidic nitrone and a method of manufacturing a data storage media comprising mixing the amidic nitrone, as a photochromic dye, with an organic material or an inorganic material to form a holographic composition and molding the holographic composition into holographic data storage media. Disclosed herein too is an article comprising a photochromic dye and an organic material, wherein the article is used as a data storage media. Disclosed herein too is a method for recording information comprising irradiating an article that comprises a photochromic dye, wherein the irradiation is conducted with electromagnetic energy having a wavelength of about 350 to about 1,100 nanometers; and reacting the photochromic dye.

### BACKGROUND

The present disclosure relates to media for recording and storing holograms, and more specifically to holographic storage media that include amidic nitrone compounds useful as photoreactive active dyes.

Holograms are typically formed by interference fringes in a holographic storage medium that diffract light in a pattern to create a readable or viewable hologram. A variety of techniques have been used to create different types of holograms. Volume holograms are an increasingly popular mechanism for the authentication of genuine articles, whether it is for security purposes or for brand protection. Volume holograms can also be used for other purposes, such as data storage, or for decorative, illustrative, or artistic purposes. The use of volume holograms for authentication purposes is driven primarily by the relative difficulty with which they can be duplicated. Volume holograms are created by interfering two coherent beams of light to create an interference pattern and storing that pattern in a holographic recording medium. Information or imagery can be stored in a hologram by imparting the data or image to one of the two coherent beams prior to their interference. The hologram can be read out by illuminating it with a beam of light matching the geometry and wavelength of either of the two original beams used to create the hologram and any data or images stored in the hologram will be displayed. As a result of the complex methods required to record holograms, their use for authentication can be seen on articles such as credit cards, software, passports, clothing, and the like.

Early holographic storage media employed inorganic photorefractive crystals, such as doped or undoped lithium niobate (LiNbO₃), in which incident light creates refractive index changes. These index changes are due to the photo-induced creation and subsequent trapping of electrons leading to an induced internal electric field that ultimately modifies the index through a linear electro-optic effect. However, LiNbO₃ is expensive, exhibits relatively poor efficiency, fades over time, and requires thick crystals to observe any significant index changes.

More recent work has led to the development of polymers that can sustain larger refractive index changes owing to optically induced polymerization processes. These materials, which are referred to as photopolymers, have significantly improved optical sensitivity and efficiency relative to LiNbO₃ and its variants. Photopolymer holographic recording media (as disclosed in e.g., U.S. Pat. No. 7,824,822 B2, U.S. Pat. No. 7,704,643 B2, U.S. Pat. No. 4,996,120 A, U.S. Pat. No. 5,013,632 A). “Single-chemistry” photopolymer systems have been employed, wherein the media comprise a homogeneous mixture of at least one photoactive polymerizable liquid monomer or oligomer, an initiator, an inert polymeric filler, and optionally a sensitizer. Since it initially has a large fraction of the mixture in monomeric or oligomeric form, the medium can have a gel-like consistency that necessitates an ultraviolet (UV) curing step to provide form and stability. Unfortunately, the UV curing step can consume a large portion of the photoactive monomer or oligomer, leaving significantly less photoactive monomer or oligomer available for data storage. Furthermore, even under highly controlled curing conditions, the UV curing step can often result in variable degrees of polymerization and, consequently, poor uniformity among media samples.

Other types of media and recording techniques have also been used to generate volume holograms. For example, dichromated gelatin, liquid crystal materials, photographic emulsions, and others as disclosed in P. Hariharan, Optical Holography—Principles, techniques, and applications 2^{nd} ed., Cambridge University Press, 1996, can all be used to generate volume holograms.

Another type of holographic recording medium utilizes a photoreactive dye dispersed in a polymer binder. This approach offers a number of advantages. Unlike many other volume holographic recording media, photoreactive dye-based media can be used with a variety of different types of polymer binders such as thermoplastics that can provide a number of beneficial physical properties specific to particular class(es) of thermoplastics (e.g., polycarbonates), such as toughness, optical clarity, scratch resistance, strength, flexibility, and the like, as well as ease of fabricating holographic articles using conventional thermoplastic fabrication techniques. Different classes of thermoplastics have different combinations of advantages and disadvantages, which can be leveraged for the desired final application.

Photoreactive dyes are compounds that undergo a light induced chemical reaction when exposed to a wavelength within the absorption range of the dye to form at least one photoproduct. This reaction can be a photodecomposition reaction, such as oxidation, reduction, or bond breaking to form smaller constituents, or a molecular rearrangement, such as a sigmatropic rearrangement, or addition reactions including pericyclic cycloadditions. The light exposure-induced chemical reaction causes a difference in refractive index in the holographic recording medium between exposed and unexposed portions of the medium, which allows for light diffracting interference fringe patterns to be formed when the medium is exposed to mutually coherent interfering signal and reference light sources.

As mentioned above, photoreactive dyes for use in holographic recording media can be used in various polymer materials. However the choice of polymer can be limited to polymers with processing temperatures (e.g., for extrusion or injection molding of thermoplastics) at which the photoreactive dye is stable. Certain nitrone compounds have been disclosed as photoreactive dyes for holographic applications, and offer beneficial optical properties for holographic purposes like high refractive index (RI), high quantum efficiency (QE), and diffraction efficiency (DE). See, for example, US 2006/0073392 A1, the disclosure of which is incorporated by reference herein in its entirety. However, there continues to be a need for photoreactive dyes that are stable at high temperatures while providing other beneficial properties for holographic recording purposes.

### SUMMARY

Disclosed herein is a holographic storage medium comprising a polymer binder and a nitrone compound according to the formula:

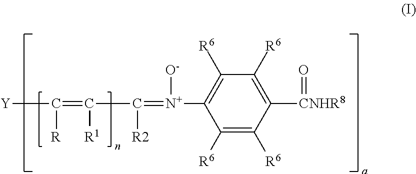

wherein Y is a monovalent or multivalent C₂-C₃₀ organic radical; each of R, R¹, and R² is independently hydrogen, an C₁-C₈ aliphatic, or a C₆-C₁₃ aromatic radical; each R⁶ is independently hydrogen, halo, cyano, nitro, a C₁-C₈ aliphatic radical, or a C₆-C₁₃ aromatic radical; R⁸ is hydrogen, a C₁-C₈ aliphatic radical, a C₆-C₈ cycloalkyl radical, or a C₆-C₁₃ aromatic radical; a is an integer of 1 to 4; and n is an integer of 0 to 4.

Disclosed herein too is an article comprising an amidic nitrone photochromic dye and an organic material, wherein the article is used as a data storage media.

Disclosed herein in addition, is a method for recording information comprising irradiating an article that comprises an amidic nitrone photochromic dye; wherein the irradiation is conducted with electromagnetic energy having a wavelength of about 350 to about 1,100 nanometers; and reacting the photochromic dye.

Disclosed herein too is a method for using a holographic data storage media comprising irradiating an article that comprises an amidic nitrone photochromic dye; wherein the irradiation is conducted with electromagnetic energy having a first wavelength and wherein the irradiating that is conducted at the first wavelength facilitates the storage of data; reacting the photochromic dye; and irradiating the article at a second wavelength to read the data.

Disclosed herein too is a method of manufacturing a holographic data storage media comprising disposing a layer of a photoactive material upon a surface of a first film; wherein the photoactive material comprises an amidic nitrone photochromic dye; and disposing a second film upon a surface of the photoactive material opposed to the surface in contact with the first film.

Amidic nitrones because of their high decomposition temperatures are suitable for use in thermoplastic compositions used in forming (e.g., by injection molding) photoactive components for holographic applications. Injection molding in holographic applications improves the processing of such holographic media and allows the use of polycarbonates and other high T_{g} (glass transition temperature) thermoplastic resins.

### DETAILED DESCRIPTION

As noted, holographic data storage relies upon the introduction of localized variations in the refractive index of the optically transparent substrate comprising the photoreactive dye as a means of storing holograms. The refractive index within an individual volume element of the optically transparent substrate can be constant throughout the volume element, as in the case of a volume element that has not been exposed to electromagnetic radiation, or in the case of a volume element in which the photoreactive dye has been reacted to the same degree throughout the volume element. It is believed that most volume elements that have been exposed to electromagnetic radiation during the holographic data writing process will contain a complex holographic pattern, and as such, the refractive index within the volume element will vary across the volume element. In instances in which the refractive index within the volume element varies across the volume element, it is convenient to regard the volume element as having an “average refractive index” which can be compared to the refractive index of the corresponding volume element before irradiation. Thus, in one embodiment an optically readable datum comprises at least one volume element having a refractive index that is different from a corresponding volume element of the optically transparent substrate before irradiation. Data storage is achieved by locally changing the refractive index of the data storage medium in a graded fashion (continuous sinusoidal variations), rather than discrete steps, and then using the induced changes as diffractive optical elements.

In one embodiment of the invention, a holographic storage medium comprising an optically transparent substrate is provided. The optically transparent substrate can be made of materials possessing sufficient optical quality such as, low scatter, low birefringence, and negligible losses at the wavelengths of interest, to render the data stored in the holographic storage medium readable. Generally, plastic materials that exhibit these properties can be used as the substrate. However, the plastic materials should be capable of withstanding the particular processing parameters employed (e.g., inclusion of the dye, exposure to a sensitizing solvent and application of any coating or subsequent layers, and molding it into a final format) and subsequent storage conditions.

Disclosed herein are novel amidic nitrone compounds useful as photoreactive dyes and optical data storage media, containing the amidic nitrone compounds, for use in holographic data storage and retrieval. Also disclosed are methods directed to holographic storage media preparation, data storage, and data retrieval. The holographic storage media is manufactured from a holographic composition that comprises a binder composition and a photoactive material, wherein the photoactive material comprises a photochromic dye. The photochromic dye comprises an amidic nitrone. The holographic storage media can be advantageously used for data storage. The holographic storage media can also be written and read (i.e., data can be stored and retrieved respectively) using electromagnetic radiation having the same wavelength.

The amidic nitrones, due to their high decomposition temperatures, can be processed at temperatures needed for processing (e.g., molding and extruding) for a variety of thermoplastics. Without wishing to be bound by theory, it is believed that the thermal stability of the amidic nitrones is a consequence of the ability of the nitrone to undergo internal hydrogen bonding.

When a particular wavelength light is incident on the nitrone compound, the nitrone is converted to an oxaziridine.

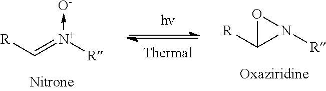

This oxaziridine normally has a different RI compared to the starting nitrone due to a change in its electronic structure. Thus, controlled bleaching of these nitrones with light to oxaziridine in polymer matrix has the ability to produce structured RI changes within a polymer matrix. This structured RI change can be engineered using laser light by the means of holograms to either write data or create optical elements in polymer matrix.

The amide is best represented as a resonance hybrid as shown below:

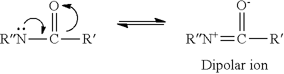

One canonical form of the amide, as shown on the right, is dipolar in character. Overall, this form lends large polarity to amide species due to large charge separation and results in strong secondary attractive forces acting in the crystal of such molecules. This also results in the formation of intermolecular hydrogen bond network between the NH group of one molecule and the O atom of the neighboring one. Without being bound by theory, it is believed that, due to this hydrogen bonding, higher energy would be required to break the crystal lattice in amidic nitrones, lending high thermal stability.

On the other hand, while increasing the stability of nitrones, such hydrogen bonding created by amide substitutions could potentially decrease the solubility of the dye compound in organic solvents and polymers. It was found, however, that the solubility of the dye chemical in a polymer matrix, for example polycarbonate, provided clear pellets during extrusion. Thus, it is believed (again without being bound by theory) that the hydrogen bonding indeed weakens at higher temperatures and high shear during extrusion, because of the enhanced kinetic motion of the molecules, and leads to solubility.

As noted above, the photoreactive compound according to the present invention is an amidic nitrone that can also be referred to as a photochromic dye. Such photochromic dyes are capable of being written and read by electromagnetic radiation in a polymeric material. In one exemplary embodiment, the photochromic dyes can be written and read using actinic radiation i.e., from about 350 to about 1,100 nanometers. In a more specific embodiment, the wavelengths at which writing and reading are accomplished may be from about 400 nanometers to about 800 nanometers. In one exemplary embodiment, the reading and writing and is accomplished at a wavelength of about 400 to about 600 nanometers. In another exemplary embodiment, the writing and reading are accomplished at a wavelength of about 400 to about 550 nanometers. In one specific exemplary embodiment, a holographic medium is adapted for writing at a wavelength of about 405 nanometers. In such a specific exemplary embodiment, reading may be conducted at a wavelength of about 532 nanometers, although viewing of holograms may be conducted at other wavelengths depending on the viewing and illumination angles, and the diffraction grating spacing and angle. Examples of photochromic dyes include diarylethenes, dinitrostilbenes and nitrones.

The amidic nitrones can be optionally used in combination with other photochromic dyes. For example, diarylethenes that can be used as photoactive materials include diarylperfluorocyclopentenes, diarylmaleic anhydrides, diarylmaleimides, or a combination comprising at least one of the foregoing diarylethenes. The diarylethenes are present as open-ring or closed-ring isomers. In general, the open ring isomers of diarylethenes have absorption bands at shorter wavelengths. Upon irradiation with ultraviolet light, new absorption bands appear at longer wavelengths, which are ascribed to the closed-ring isomers.

The amidic nitrones are of generic structure (I):

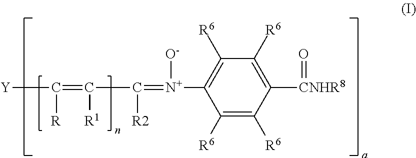

wherein Y is a monovalent or multivalent C₂-C₃₀ organic radical; each of R, R¹, and R² is independently hydrogen, an C₁-C₈ aliphatic, or a C₆-C₁₃ aromatic radical; each R⁶ is independently hydrogen, halo, cyano, nitro, a C₁-C₈ aliphatic radical, or a C₆-C₁₃ aromatic radical; R⁸ is hydrogen, a C₁-C₈ aliphatic radical, a C₆-C₈ cycloalkyl radical, or a C₆-C₁₃ aromatic radical; a is an integer of 1 to 4, more specifically 1 or 2; and n is an integer of 0 to 4. More specifically, each aromatic radical can be an aryl group, each aliphatic radical can be an alkyl group, and each cycloaliphatic radical can be a cycloalkyl group.

Specifically, when a is an integer of 2 to 4 and Y is a multivalent organic radical, Y can be a C₁-C₂₀ aliphatic radical, a C₃-C₂₀ cycloaliphatic radical, or a C₃-C₃₀ aromatic radical; and “a” is an integer from 2 to 100. In one embodiment, “a” is 2 and Y is a divalent organic radical.

In the case where Y is a monovalent radical and a is 1, Y specifically can be Z, wherein Z is (R³)_{b}-Q-R⁴— or R⁵—, wherein Q is a monovalent, divalent or trivalent substituent or linking group specifically having 1 to 8 carbon atoms; wherein each of R, R¹, R² and R³ is independently hydrogen, an aliphatic radical having 1 to 8 carbon atoms or an aromatic radical having 6 to 13 carbon atoms; n is up to 4; b is up to 3; R⁴ is an aromatic radical having 6 to 13 carbon atoms; R⁵ is an aromatic radical having 6 to 20 carbon atoms (which optionally can have substituents that contain hetero atoms, including oxygen, nitrogen or sulfur); each R⁶ is independently hydrogen, halo, cyano, nitro, an aliphatic radical having 1 to 8 carbon atoms or an aromatic radical having 6 to 13 carbon atoms; R⁸ is an aliphatic radical having 1 to 8 carbon atoms, a cycloaliphatic radical having 6 to 8 carbon atoms, or an aromatic radical having 6 to 13 carbon atoms; n is an integer of 0 to 4; and b is an integer of 0 to 3.

For example, Q herein can be an R group, as defined above, substituted with up to three R groups. When b is not zero, Q is a linking group. Again and in general in this application, more specifically each aromatic radical can be an aryl group, each aliphatic group can be an alkyl group, and each cycloaliphatic radical can be a cycloalkyl group.

One class of nitrones, according to an exemplary embodiment of the invention, comprises an aryl nitrone represented by the formula (II):

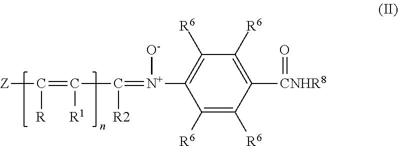

wherein Z is (R³)_{b}-Q-R⁴— or R⁵—; Q is a monovalent, divalent or trivalent substituent or linking group; wherein each of R, R¹, R², and R³ is independently hydrogen, an aliphatic radical having 1 to 8 carbon atoms or an aromatic radical having 6 to 13 carbon atoms; R⁴ is an aromatic radical having 6 to 13 carbon atoms; b is an integer of 0 to 3; n is an integer of 0 to 4; R⁴ is an aromatic radical having 6 to 13 carbon atoms; R⁵ is an aromatic radical having 6 to 20 carbon atoms; each R⁶ is independently hydrogen, halo, cyano, nitro, an aliphatic radical having 1 to 8 carbon atoms or an aromatic radical having 6 to 13 carbon atoms; and R⁸ is an aliphatic radical having 1 to 8 carbon atoms, a cycloaliphatic radical having 6 to 8 carbon atoms, or an aromatic radical having 6 to 13 carbon atoms; n is up to 4; and b is up to 3. More specifically, aliphatic radicals can be C₁-C₈ alkyl groups and aromatic radicals can be phenyl groups that can be optionally substituted.

In a more specific embodiment of formula (II), n can be zero, R² can be hydrogen, R⁴ and R⁵ can be phenyl, and R⁸ can be a C₁-C₆ alkyl, a phenyl, or a cyclohexyl group.

As can be seen from formula (II), the nitrones can be α-aryl-N-arylnitrones or conjugated analogs thereof in which the conjugation is between the aryl group and an α-carbon atom. In addition to the amido group, the N-aryl group can be further substituted, for example, by a dialkylamino group in which the alkyl groups contain 1 to 4 carbon atoms. In one embodiment, R² and R⁶ are hydrogen and R⁸ is phenyl.

Suitable examples of nitrones are α-(4-ethylamidophenyl)-N-phenylnitrone; α-(4-ethylamidophenyl)-N-(4-chlorophenyl)-nitrone, α-(4-ethylamidophenyl)-N-(3,4-dichlorophenyl)-nitrone, α-(4-ethylamidophenyl)-N-(4-carbethoxyphenyl)-nitrone, α-(4-ethylamidophenyl)-N-(4-acetylphenyl)-nitrone, α-(4-ethylamidophenyl)-N-(4-cyanophenyl)-nitrone, α-(4-ethylamidophenyl)-N-(4-cyanophenyl)nitrone, α-[2-(1,1-di(4-ethylamidophenyl)ethenyl)]-N-phenylnitrone, α-[2-(1-(4-ethylamidophenyl)propenyl)]-N-phenylnitrone, or the like, or a combination comprising at least one of the foregoing nitrones. An exemplary aryl nitrone is α-(4-ethylamidophenyl)-N-phenylnitrone.

In another embodiment, the photoreactive dye is a compound (III) comprising at least two nitrone groups:

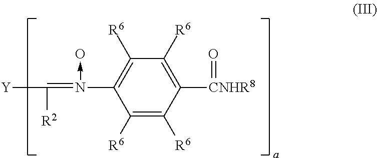

wherein Y is a C₁-C₂₀ aliphatic radical, a C₃-C₂₀ cycloaliphatic radical, or a C₃-C₃₀ aromatic radical; a is an integer from 2 to 4; and R², R⁶, and R⁸ are as defined for formula (I).

Representative polynitrone compounds are illustrated in Table I. One of ordinary skill in the art will appreciate the relationship between generic formulas above and the individual structures of entries 1a-1e of Table I.

TABLE 1

| Example | Structure |
|---|---|
| 1a | 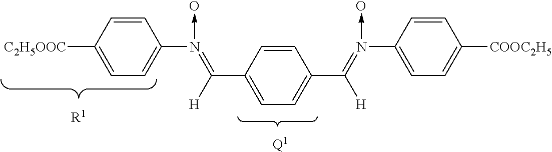 |
| 1b | 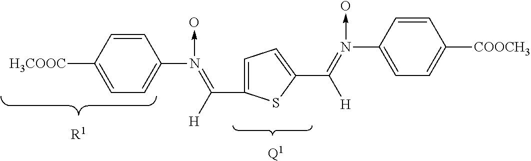 |
| 1c | 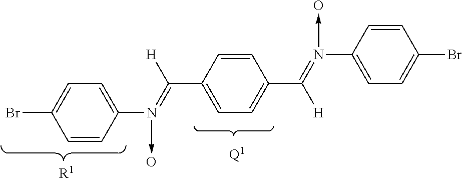 |
| 1d | 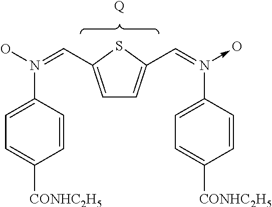 |
| 1e | 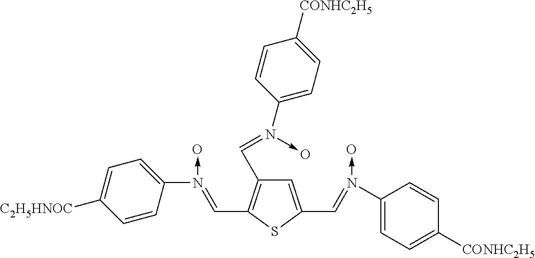 |

In one embodiment, the photoreactive dye is a furan-, thiophene-, or pyrole-containing polynitrone having formula (IV)

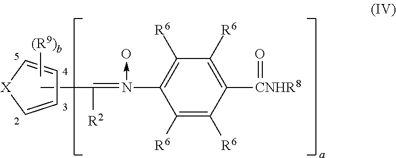

wherein a is an integer from 2 to 4; X is O, S, or NH, R², R⁶ and R⁸ are as defined above; R⁹ is independently at each occurrence a halogen, a hydrogen, a deuterium, a C₁-C₂₀ aliphatic radical, a C₃-C₂₀ cycloaliphatic radical, or a C₃-C₃₀ aromatic radical; and b is an integer from 0 to 2, wherein a+b=4. For convenience in formula (IV) 6, the positions of the carbon atoms in the thiophene are labeled 2-5.

In one exemplary embodiment, a=2 and b=0. The nitrone moieties wherein X, R², R⁶, and R⁸ are defined above, can be attached to positions 2 and 3 of the furan, thiophene, or pyrole moiety to provide dye formula (V):

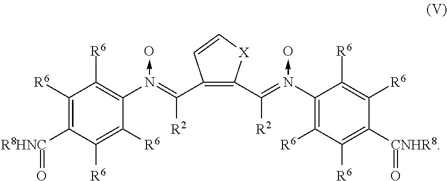

Alternatively, the nitrone moieties can be attached to positions 2 and 4, or 2 and 5, or 3 and 4 of the furan, thiophene, or pyrole moiety to provide the corresponding dyes.

In another embodiment, a=2 and b=2, and nitrone moieties are attached to positions 2 and 3, 2 and 4, or 2 and 5 of the furan, thiophene, or pyrole moiety, and each R⁹ is attached to the remaining positions, that is, 4 and 5, 3 and 5, or 3 and 4, respectively. A specific embodiment is the furan-, thiophene-, or pyrole-containing dinitrone formula (VI)

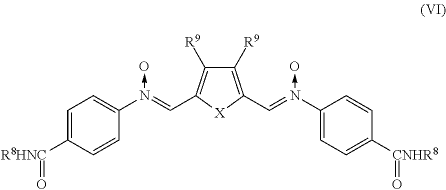

wherein X is as defined above, each R⁸ is independently at each occurrence a C₁-C₂₀ aliphatic radical, a C₃-C₂₀ cycloaliphatic radical, or a C₃-C₃₀ aromatic radical, and R⁹ is independently at each occurrence a halogen, a C₁-C₂₀ aliphatic radical, a C₃-C₂₀ cycloaliphatic radical, or a C₃-C₃₀ aromatic radical.

In another embodiment, a furan-, thiophene-, or pyrole-containing dinitrone has formula (VII)

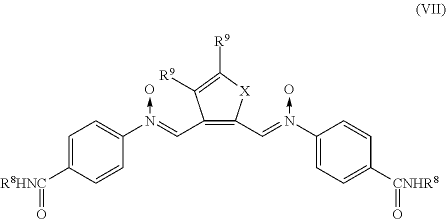

wherein X is as defined above, each R⁸ is independently at each occurrence a C₁-C₂₀ aliphatic radical, a C₃-C₂₀ cycloaliphatic radical, or a C₃-C₃₀ aromatic radical, and R⁹ is independently at each occurrence a halogen, a C₁-C₂₀ aliphatic radical, a C₃-C₂₀ cycloaliphatic radical, or a C₃-C₃₀ aromatic radical.

An exemplary preparation of nitrones can include the following steps. The first step is the preparation of nitro derivatives containing carbon amide derivatives, as follows:

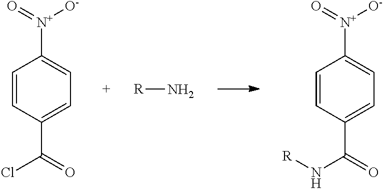

The second step is the preparation of an hydroxylamine, as follows:

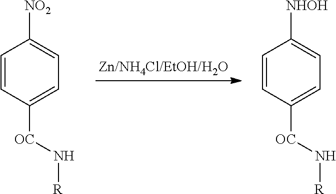

Other methods are known for the preparation of hydroxylamine from a nitro compound. For example, in one method, an aromatic or aliphatic nitro compound is converted into a corresponding hydroxylamine using zinc and ammonium chloride in aqueous alcohol and then reacted with an aromatic or thiophene dicarboxaldehye. In another method, an in situ process, the following compounds are reacted in one pot: a nitro compound, an aromatic or thiophene dicarboxaldehyde, zinc, and acetic acid. In still another method (a hydrogenation method), nitro compounds can be converted to their hydroxylamine derivative using hydrogen and Pd/C in the presence of DMSO.

The third step is condensation of the hydroxylamine derivative with an aldehyde. Dinitrones can be prepared employing the following reaction.

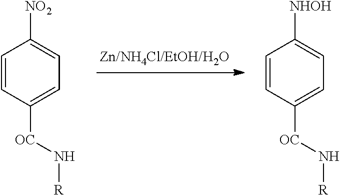

R¹=

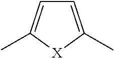

where X is O, S, or NH; or R¹ is divalent aromatic
R=H, alkyl, alkyl ester, etc.

Mono-nitrones can be prepared by the following reaction.

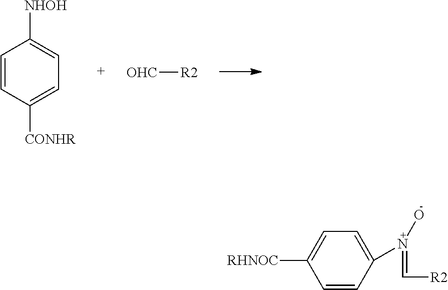

R2=H, alkyl, phenyl, .etc

Upon exposure to electromagnetic radiation, nitrones of formula (I) undergo unimolecular cyclization to an oxaziridine of formula (IA):

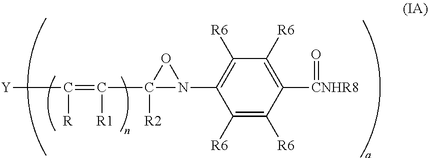

wherein R, R¹, R², R⁶, R⁸, a, and Y have the same meaning as denoted above for (I).

Thus, one aspect of the invention relates to an article or photoproduct that comprises an oxaziridine compound. In some embodiments, the photocyclization of the photoreactive nitrone dye to an oxaziridine photoproduct proceeds with a high quantum efficiency, and a large refractive index change. Typically, the photocyclization is induced in only a portion of the total amount of the photoreactive nitrone dye present in a given volume element, thus providing a refractive index contrast between the unconverted dye and the oxaziridine photo-product, and providing the concentration variations of the photo-product corresponding to the holographic interference pattern, and constituting the optically readable datum.

The binder composition can include inorganic material(s), organic material(s), or a combination of inorganic material(s) with organic material(s), wherein the binder has sufficient deformability (e.g., elasticity and/or plasticity) to enable the desired number of deformation states (e.g., number of different deformation ratios) for the desired recording. The binder should be an optically transparent material, e.g., a material that will not interfere with the reading or writing of the hologram. As used herein, the term “optically transparent” means that an article (e.g., layer) or a material capable of transmitting a substantial portion of incident light, wherein a substantial portion can be greater than or equal to 70% of the incident light. The optical transparency of the layer may depend on the material and the thickness of the layer. The optically transparent holographic layer may also be referred to as a holographic layer.

Exemplary organic materials include optically transparent organic polymer(s) that are elastically deformable. In one embodiment, the binder composition comprises elastomeric material(s) (e.g., those which provide compressibility to the holographic medium). Exemplary elastomeric materials include those derived from olefins, monovinyl aromatic monomers, acrylic and methacrylic acids and their ester derivatives, as well as conjugated dienes. The polymers formed from conjugated dienes can be fully or partially hydrogenated. The elastomeric materials can be in the form of homopolymers or copolymers, including random, block, radial block, graft, and core-shell copolymers. Combinations of elastomeric materials can be used.

Possible elastomeric materials include thermoplastic elastomeric polyesters (commonly known as TPE) include polyetheresters such as poly(alkylene terephthalates) (particularly poly[ethylene terephthalate] and poly[butylene terephthalate]), e.g., containing soft-block segments of poly(alkylene oxide), particularly segments of poly(ethylene oxide) and poly(butylene oxide); and polyesteramides such as those synthesized by the condensation of an aromatic diisocyanate with dicarboxylic acids and a carboxylic acid-terminated polyester or polyether prepolymer. One example of an elastomeric material is a modified graft copolymer comprising (i) an elastomeric (i.e., rubbery) polymer substrate having a glass transition temperature (Tg) less than 10° C., more specifically less than −10° C., or more specifically −200° to −80° C., and (ii) a rigid polymeric superstrate grafted to the elastomeric polymer substrate. Exemplary materials for use as the elastomeric phase include, for example, conjugated diene rubbers, for example polybutadiene and polyisoprene; copolymers of a conjugated diene with less than 50 wt % of a copolymerizable monomer, for example a monovinylic compound such as styrene, acrylonitrile, n-butyl acrylate, or ethyl acrylate; olefin rubbers such as ethylene propylene copolymers (EPR) or ethylene-propylene-diene monomer rubbers (EPDM);
ethylene-vinyl acetate rubbers; silicone rubbers; elastomeric C₁₋₈ alkyl(meth)acrylates; elastomeric copolymers of C₁₋₈ alkyl(meth)acrylates with butadiene and/or styrene; or combinations comprising at least one of the foregoing elastomers. Exemplary materials for use as the rigid phase include, for example, monovinyl aromatic monomers such as styrene and alpha-methyl styrene, and monovinylic monomers such as acrylonitrile, acrylic acid, methacrylic acid, and the C₁-C₆ esters of acrylic acid and methacrylic acid, specifically methyl methacrylate. As used herein, the term “(meth)acrylate” encompasses both acrylate and methacrylate groups.

Specific exemplary elastomer-modified graft copolymers include those formed from styrene-butadiene-styrene (SBS), styrene-butadiene rubber (SBR), styrene-ethylene-butadiene-styrene (SEBS), ABS (acrylonitrile-butadiene-styrene), acrylonitrile-ethylene-propylene-diene-styrene (AES), styrene-isoprene-styrene (SIS), methyl methacrylate-butadiene-styrene (MBS), and styrene-acrylonitrile (SAN).

Exemplary organic materials that can also be employed as the binder composition are optically transparent organic polymers. The organic polymer can be thermoplastic polymer(s), thermosetting polymer(s), or a combination comprising at least one of the foregoing polymers. The organic polymers can be oligomers, polymers, dendrimers, ionomers, copolymers such as for example, block copolymers, random copolymers, graft copolymers, star block copolymers; or the like, or a combination comprising at least one of the foregoing polymers. Exemplary thermoplastic organic polymers that can be used in the binder composition include, without limitation, polyacrylates, polymethacrylates, polyesters (e.g., cycloaliphatic polyesters, resorcinol arylate polyester, and so forth), polyolefins, polycarbonates, polystyrenes, polyamideimides, polyarylates, polyarylsulfones, polyethersulfones, polyphenylene sulfides, polysulfones, polyimides, polyetherimides, polyetherketones, polyether etherketones, polyether ketone ketones, polysiloxanes, polyurethanes, polyethers, polyether amides, polyether esters, or the like, or a combination comprising at least one of the foregoing thermoplastic polymers (either in admixture or co- or graft-polymerized), such as polycarbonate and polyester.

Exemplary polymeric binders are described herein as “transparent”. Of course, this does not mean that the polymeric binder does not absorb any light of any wavelength. Exemplary polymeric binders need only be reasonably transparent in wavelengths for exposure and viewing of a holographic image so as to not unduly interfere with the formation and viewing of the image. In an exemplary embodiment, the polymer binder has an absorbance in the relevant wavelength ranges of less than 0.2. In another exemplary embodiment, the polymer binder has an absorbance in the relevant wavelength ranges of less than 0.1. In yet another exemplary embodiment, the polymer binder has an absorbance in the relevant wavelength ranges of less than 0.01. Organic polymers that are not transparent to electromagnetic radiation can also be used in the binder composition if they can be modified to become transparent. For example, polyolefins are not normally optically transparent because of the presence of large crystallites and/or spherulites. However, by copolymerizing polyolefins, they can be segregated into nanometer-sized domains that cause the copolymer to be optically transparent.

In one embodiment, the organic polymer and photochromic dye can be chemically attached. The photochromic dye can be attached to the backbone of the polymer. In another embodiment, the photochromic dye can be attached to the polymer backbone as a substituent. The chemical attachment can include covalent bonding, ionic bonding, or the like.

Examples of cycloaliphatic polyesters for use in the binder composition are those that are characterized by optical transparency, improved weatherability and low water absorption. It is also generally desirable that the cycloaliphatic polyesters have good melt compatibility with the polycarbonate resins since the polyesters can be mixed with the polycarbonate resins for use in the binder composition. Cycloaliphatic polyesters are generally prepared by reaction of a diol (e.g., straight chain or branched alkane diols, and those containing from 2 to 12 carbon atoms) with a dibasic acid or an acid derivative.

Polyarylates that can be used in the binder composition refer to polyesters of aromatic dicarboxylic acids and bisphenols. Polyarylate copolymers include carbonate linkages in addition to the aryl ester linkages, known as polyester-carbonates. These aryl esters may be used alone or in combination with each other or more particularly in combination with bisphenol polycarbonates. These organic polymers can be prepared, for example, in solution or by melt polymerization from aromatic dicarboxylic acids or their ester forming derivatives and bisphenols and their derivatives.

Blends of organic polymers may also be used as the binder composition for the holographic devices. Specifically, organic polymer blends can include polycarbonate (PC)-poly(1,4-cyclohexane-dimethanol-1,4-cyclohexanedicarboxylate) (PCCD), PC-poly(cyclohexanedimethanol-co-ethylene terephthalate) (PETG), PC-polyethylene terephthalate (PET), PC-polybutylene terephthalate (PBT), PC-polymethylmethacrylate (PMMA), PC-PCCD-PETG, resorcinol aryl polyester-PCCD, resorcinol aryl polyester-PETG, PC-resorcinol aryl polyester, resorcinol aryl polyester-polymethylmethacrylate (PMMA), resorcinol aryl polyester-PCCD-PETG, or the like, or a combination comprising at least one of the foregoing.

Binary blends, ternary blends and blends having more than three resins may also be used in the polymeric alloys. When a binary blend or ternary blend is used in the polymeric alloy, one of the polymeric resins in the alloy may comprise about 1 to about 99 weight percent (wt %) based on the total weight of the composition. Within this range, it is generally desirable to have the one of the polymeric resins in an amount greater than or equal to about 20, preferably greater than or equal to about 30 and more preferably greater than or equal to about 40 wt %, based on the total weight of the composition. Also desirable within this range, is an amount of less than or equal to about 90, preferably less than or equal to about 80 and more preferably less than or equal to about 60 wt % based on the total weight of the composition. When ternary blends of blends having more than three polymeric resins are used, the various polymeric resins may be present in any desirable weight ratio.

Exemplary thermosetting polymers that may be used in the binder composition include, without limitation, polysiloxanes, phenolics, polyurethanes, epoxies, polyesters, polyamides, polyacrylates, polymethacrylates, or the like, or a combination comprising at least one of the foregoing thermosetting polymers. In one embodiment, the organic material can be a precursor to a thermosetting polymer.

In addition to binder, the optically transparent substrate can comprise additional components such as heat stabilizers; antioxidants; light stabilizers; plasticizers; antistatic agents; mold releasing agents; additional resins; binders, blowing agents; and the like, as well as combinations of the foregoing additives.

In one embodiment, as noted above, in a method for storing holographic data, an optically transparent substrate containing the nitrone photochromic dye is irradiated with a holographic interference pattern, wherein the pattern has a first wavelength and an intensity both sufficient to convert, within a volume element of the substrate, at least some of the photoreactive dye into a photo-product, and producing within the irradiated volume element concentration variations of the photoproduct corresponding to the holographic interference pattern, thereby producing an optically readable datum corresponding to the volume element. The optically readable datum is stored in the optically transparent substrate as a hologram patterned within at least one volume element of the optically transparent substrate.

Those skilled in the art will appreciate that the lingering photosensitivity of an unconverted (residual) photochemically reactive dye can present a problem that can adversely affect the integrity of the stored data if no step is taken to stabilize the unconverted photochemically reactive dye. In the case where the unconverted photochemically reactive dye is a nitrone, protonation of the nitrone remaining following the recording of the holographic data can provide an efficient means of preventing further conversion of the nitrone to photoproducts under the influence of, for example, a read beam or ambient light.

In one embodiment, the optically transparent substrate containing the nitrone photochromic dye is irradiated with a holographic interference pattern having a first wavelength to record data. The optically transparent substrate is then irradiated with radiation having a second wavelength to stabilize the written data, and the stabilized data can then be read using radiation having a third wavelength (e.g., a “read beam”), wherein the radiation at each step can independently have a wavelength from about 300 nm to about 1,500 nm In an embodiment, the first, second, and third wavelengths can be independently between about 300 nm and about 800 nm In one embodiment, the first wavelength (or the writing wavelength) for writing and recording the data onto the holographic data storage medium is from about 375 nm to about 450 nm. In another embodiment, the first wavelength can be from about 450 nm to about 550 nm In one embodiment, the first wavelength is in a range from about 375 nm to about 450 nm and the second wavelength is in a range from about 450 to about 1500 nm In another embodiment, the first wavelength is in a range from about 450 nm to about 550 nm and the second wavelength is in a range from about 550 to about 1500 nm In still another embodiment, the writing wavelength is such that it is shifted by 0 nm to about 400 nm from the wavelength at which the recorded data is stabilized by the action of light of the second wavelength. Exemplary wavelengths at which writing and data stabilization are accomplished are about 405 nanometers (writing) and about 532 nanometers (stabilization). The first wavelength is also sometimes referred to as the “write” wavelength.

In one embodiment, the photochromic dye after being reacted can be converted to a non-photochromic state so that any written data cannot be destroyed. The conversion of the photochromic dye to the non-photochromic state can be induced by an electric field, by a third wavelength, by a photoacid generator or by a combination comprising at least one of the foregoing.

In another embodiment of a method of manufacturing the holographic data storage media, the photoactive material is disposed upon a first film that comprises an organic polymer. The first film behaves as a substrate upon which is disposed the photoactive material. The photoactive material can be disposed upon the first film in the form of a complete or partial layer. In yet another embodiment, a second film is disposed upon a surface of the photoactive material opposed to the surface in contact with the first film. The first and the second films can be molded or cast from solution. The second film can be disposed upon the surface of the photoactive material by molding. The photoactive material is then coated onto the surface of the first film or the surface of the second film or upon the opposing surfaces of both the first film and the second film. Examples of processes by which the photoactive material can be coated onto the surface of the film are by brush painting, dip coating, spray painting, spin coating, or the like.

When a photochromic material is disposed upon a film to form the holographic data storage as described above, it is generally desirable to have the film having a thickness of about 1 to about 100,000 micrometers (μm). In one embodiment, it is desirable to have a thickness of about 2 to about 10,000 μm. In another embodiment, it is desirable to have a thickness of about 3 to about 1,000 μm. In yet another embodiment, it is desirable to have a thickness of about 7 to about 500 μm.

In another embodiment of a method of manufacturing the holographic data storage media, the photoactive material can be incorporated into the organic polymer in a mixing process to form a data storage composition. Following the mixing process, the data storage composition can be molded into an article that can be used as holographic data storage media. Examples of molding can include injection molding, blow molding, compression molding, vacuum forming, or the like. The injection molded article can have any geometry. Examples of suitable geometries are circular discs, square shaped plates, polygonal shapes, or the like.

The mixing processes by which the photoactive material can be incorporated into the organic polymer involves the use of shear force, extensional force, compressive force, ultrasonic energy, electromagnetic energy, thermal energy or combinations comprising at least one of the foregoing forces or forms of energy and is conducted in equipment wherein the aforementioned forces are exerted by a single screw, multiple screws, intermeshing co-rotating or counter rotating screws, non-intermeshing co-rotating or counter rotating screws, reciprocating screws, screws with pins, screws with screens, barrels with pins, rolls, rams, helical rotors, baffles, or combinations comprising at least one of the foregoing.

The mixing can be conducted in machines such as a single or multiple screw extruder, a Buss kneader, a Henschel, a helicone, an Eirich mixer, a Ross mixer, a Banbury, a roll mill, molding machines such as injection molding machines, vacuum forming machines, blow molding machine, or then like, or a combination comprising at least one of the foregoing machines.

A holographic composition containing the photochromic amidic nitrone dye can comprise about 0.1 to about 50 weight percent (wt %), based on the total weight of the holographic composition. In one embodiment, the holographic composition comprises about 1 to about 40 wt %, based upon the total weight of the holographic composition. In another embodiment, the holographic composition comprises about 2 to about 20 wt %, based upon the total weight of the holographic composition. In yet another embodiment, the holographic composition comprises about 3 to about 10 wt %, based upon the total weight of the holographic composition.

In one embodiment, a data storage composition comprising a photoreactive nitrone and a thermoplastic polymer is injection molded to form an article that can be used for producing holographic data storage media. The injection-molded article can have any geometry. Examples of suitable geometries include circular discs, square shaped plates, polygonal shapes, or the like. The thickness of the articles can vary, from being at least 100 micrometers in an embodiment, and at least 250 micrometers in another embodiment. A thickness of at least 250 micrometers is useful in producing holographic data storage disks that are comparable to the thickness of current digital storage discs. In some embodiments, the thickness can vary from about 100 micrometers to about 5 centimeters. For example, for use as a DVD or CD storage device typical thickness is about 600 micrometers to about 1.2 millimeters.

After the molding of the data storage media the data can be stored onto the media by irradiating the media with electromagnetic energy having a first wavelength. The irradiation facilitates the conversion of the open form of the isomer to the closed form of the isomer (cyclization) of the photochromic dye thereby creating a hologram into which the data is encoded.

The holographic materials as described herein can be exposed to form holograms using any of a number of exposure setups, which are well-known in the art. A simple exposure setup, for example, is described in US 2006/0073392 A1, the disclosure of which is incorporated herein by reference in its entirety.

The present disclosure is illustrated by the following non-limiting examples.

### EXAMPLES

### Materials:

The aldehydes and nitro compounds required for nitrone preparation were procured from Aldrich Chemicals and were used without further purifications.

### Analytical Methods:

Proton NMR spectra for all the starting materials and products described herein were measured using a 300 MHz Bruker NMR spectrometer and d₆-dimethylsulfoxide as solvent.

Compounds were further characterized by a liquid chromatograph-mass spectrometer (LC-MS) system, comprising a liquid chromatograph and a Quattro Ultima Pt mass spectrometer. An Xterra C18 (50 mm×4.6 mm; 5 microns) column was used for the separating the components by liquid chromatography. The separated components were then analyzed by mass spectrometry.

Ultraviolet-visible (UV-VIS) spectra were recorded using a double beam Perkin-Elmer Lambda 900 UV-VIS-NIR spectrophotometer.

Infrared (IR) spectra were obtained using a Perkin Elmer Spectrum GX series instrument by employing the attenuated total reflectance.

DSC experiments were performed to study the thermal behavior of nitrones especially for melting or decomposition temperature. The melting or decomposition temperatures were measured in presence nitrogen with a heating rate of 10° C./min using DSCQ10 (TA) instrument.

Purity analysis, for quantification of compound survival in extruded pellets, was carried out by normal phase high performance liquid chromatographic method using chloroform as eluent. The detailed experiment conditions are given as follows: column: 250 mm silica, temperature: 30° C., flow rate: 1 ml/min, detector force PDA, detection wavelength 280 nm, Instrument: Shimadzu.

### Method of Extrusion:

Different loading level of nitrones (identified by compound number references as set forth in Tables 4 and 5 below) were used to make samples for this study. The compositions of samples are listed in Table 2

TABLE 2

| Formulations in parts per hundred | | | | | | |
|---|---|---|---|---|---|---|
| Material | | Batch Number | | | | |
| | 1 | 2 | 3 | 4 | 5 | 6 |
| Low Tg Poly- | 100 | 100 | 100 | 100 | 100 | 100 |
| carbonate | | | | | | |
| IRGAFOS-168 | 0.06 | 0.06 | 0.06 | 0.06 | 0.06 | 0.06 |
| stabilizer* | | | | | | |
| PETS mold | 0.26 | 0.26 | 0.26 | 0.26 | 0.26 | 0.26 |
| release agent | | | | | | |
| Compound 8 | 0.5 | 1 | 3 | | | |
| Compound 10 | | | | 0.5 | 3 | |
| Compound 12 | | | | | | 2 |
| Total | 100.82 | 101.32 | 103.32 | 100.82 | 103.32 | 102.32 |

*Heat stabilizer from Ciba-Geigy

A mixture of 1 kg polycarbonate PC 105 with the required amount of amidic nitrone was taken in a zip-lock polyethylene bag and shaken vigorously for about 3-4 min The resulting mixture was then compounded by using a W&P ZSK-25 Mega Compounder under vacuum under the conditions specified in Table 3 to produce polymer pellets.

TABLE 3

| | |
|---|---|
| Feed zone temperature (° C.) | 125 |
| Zone 1 temperature (° C.) | 220 |
| Zone 2 temperature (° C.) | 230 |
| Zone 3 temperature (° C.) | 250 |
| Zone 4 temperature (° C.) | 260 |
| Throat/Die temperature (° C.) | 270 |
| Screw speed (Revolutions per minute) | 300 |
| Torque (Nm) | 50-60 |

### Example 1

The compound 4 (2,6-diisopropylamidoxy)phenyl hydroxylamine was prepared, as follows. In a first step, the following reaction was used:

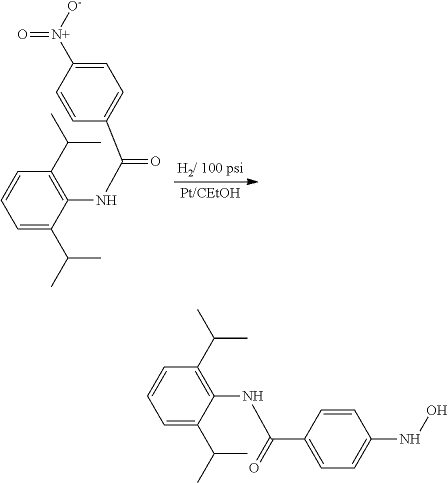 [SMILES: CC(C)c1cccc(C(C)C)c1NC(=O)c1ccc([N+](=O)[O-])cc1 ; CC(C)c1cccc(C(C)C)c1NC(=O)c1ccc(NO)cc1]

In a Parr hydrogenater (volume—100 ml) charged 3.26 g (10 mmol) of N-(2,6-diisopropyl-phenyl)-4-nitro-benzamide, 25 mg of 10% Pt on carbon, 0.5 mL of DMSO and 30 mL of absolute ethanol. The system was flushed 3 times with 50 psi of hydrogen and finally charged to 100 psi with hydrogen. The reaction mixture was stirred until hydrogen uptake ceased (˜3 hr). The Pt/C was filtered from the reaction mixture to get the hydroxylamine solution. This hydroxylamine was directly used for the next step formation of the dinitrone without characterization. (The hydroxylamine can also be isolated by removal of the ethanol. If the starting nitro compound does not have a p-electron withdrawing group, then hydrogenation is stopped after two equivalents of hydrogen have reacted in order to minimize the amount of over reduction to the amine.)

In a second step, the amidic dinitrone was prepared, as follows, using the following reaction.

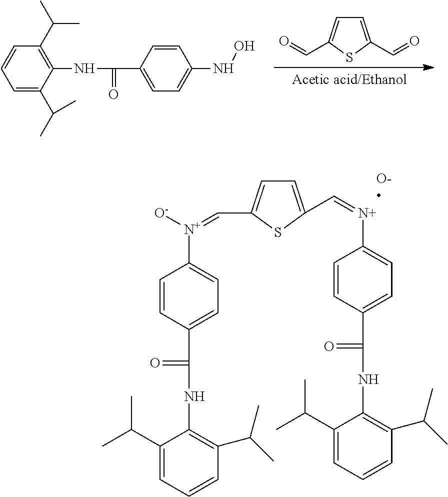

To the hydroxylamine solution is further added 400 mg (2.8 mmol) 2,5-thiophenedialdehyde and 10 ml of acetic acid in the second step of the nitrone synthesis. The reaction mixture is further stirred for 10 hrs at room temperature.

The dinitrone formed is filtered and washed with 200 ml of ethanol, 200 ml water, then with 200 ml of dichloromethane to remove the excess hydroxylamine and the other impurities formed. Finally, a wash with 200 ml hexane is given to the dinitrone, which is then dried to get 1.5 g (41%) of the pure (90%) dinitrone. The dinitrone formation was confirmed by LC-MS and NMR, also purity by HPLC.

### Example 2

The compound 2,5-thiophene bis N,N-dibutylcarbonimidophenyl dinitrone was prepared as follows.

In a first step, 4-nitro-phenyl-N,N-dibutylcarbonimide was prepared, as follows, using the following reaction.

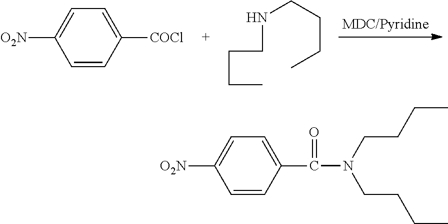

To the 250 mL, 3-necked flask was added 5.2 gm Dibutylamine, 100 ml dichloromethane, and 3.0 g pyridine. The contents were stirred for 10 minutes. To this mixture, 7.1 g 4-nitro-benzoyl chloride was added during 10 minutes and the mixture stirred at reflux for 2 hrs. The reaction mixture was then cooled and then washed with 20% aqueous ammonium hydroxide. The organic layer was washed with 1 N hydrochloric acid and brine, dried over anhydrous sodium sulphate and evaporated to give crude product =11.2 gm (˜93% yield).

In a second step, the 4-hydroxyamino-phenyl-N,N-dibutylcarbonimide was prepared, as follows, using the following reaction.

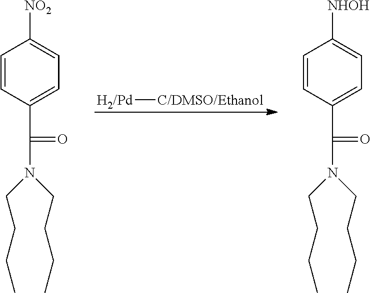

Subsequently, the 2,5-thiophene bis-N,N-dibutylcarbonamidephenyl dinitrone was prepared as follows, using the following reaction:

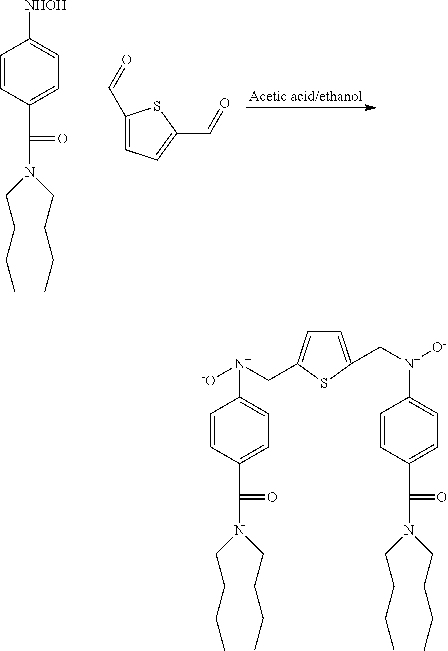

In a Parr hydrogenater (volume—100 ml) charged 2.78 g (10 mmol) of N,N-dibutyl-4-nitro-benzamide, 30 mg of 10% Pd on carbon, 0.5 g of DMSO and 30 mL of absolute ethanol. The system was flushed 3 times with 50 psi of hydrogen and finally charged to 250 psi with hydrogen. The reaction mixture was stirred until hydrogen uptake ceased (˜3 hr. and around 60-70 psi of hydrogen consumed). The Pd/C was filtered from the reaction mixture to get the hydroxylamine solution. The N,N-dibutyl-4-hydroxyamino-benzamide formation is confirmed by LC-MS.

To the hydroxylamine solution is further added 350 mg (2.5 mmol) 2,5-thiophenedialdehyde and 15 ml of acetic acid in the second step of the nitrone synthesis. The reaction mixture was further stirred for 10 hrs at room temperature. To the reaction mixture was added 50 ml of dichloromethane and further added 200 ml of water. The mixture was shaken well to extract all the organic part in to the dichloromethane and separated from the other fraction.

Dichloromethane was further distilled out under reduced pressure. Then 400 ml of hexane was added to the mixture and shaken well to precipitate out and obtain the product. The product was filtered and washed with hexane: dichloromethane (9.5:0.5) mixture (200 ml) to remove the excess hydroxylamine and other impurities formed (purity above 90%). The product obtained was further dried in vacuum to get 1.2 g of pure (91%) dinitrone. The dinitrone formation was confirmed by LC-MS and also purity by HPLC.

### Example 3

This example illustrates the preparation of the amidic mono nitrone compound N-(4-methylphenyl)-1-(4-phenylphenyl)methanimine oxide; N-hexylformamide.

In a first step, N-hexyl-4-nitrobenzamide was prepared, as follows, using the following reaction:

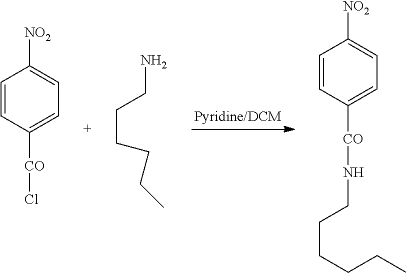

To a three-necked round-bottomed flask, equipped with reflux condenser, calcium chloride guard tube with nitrogen inlet and a magnetic stirrer, was charged dichloromethane (200 g), 4-nitro-benzoyl chloride (18.5 g, 0.1 mole) and pyridine (7.9 g, 0.1 mole). The mixture was cooled to 10-15° C. and aniline (9.5 g) added in lots by keeping the temperature below 30° C. The reaction mixture was stirred at room temperature (−25° C.) for 2 hr. Then 5% HCl solution (300 ml) was added to the reaction mixture and stirred for 30 minutes. The aqueous layer was separated out and a further 300 ml of 5% sodium hydroxide washing was given to the reaction mixture. The aqueous layer was separated out and washed with water until neutral. After this, 200 ml of hexane was added to the reaction mixture to precipitate out the product completely. The thus obtained solid product was filtered and washed with water. The solid product was dried in an oven at 70° C. Yield: 22.5 g (90%); HPLC Purity (% A): 99.0%; Mass: m/z: 251.

Next, the N-hexyl-4-hydroxylamine benzamide was prepared, as follows, using the following reaction:

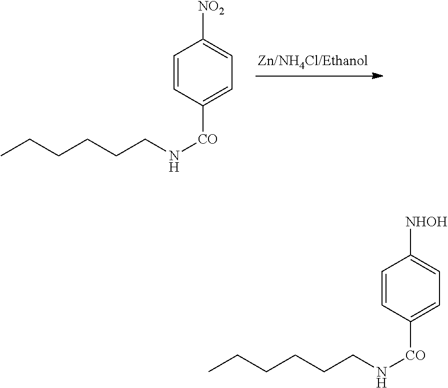

A solution of N-hexyl-4-nitrobenzamide (10 g) in warm 95% ethanol (150 ml) was mixed with ammonium chloride (2.39 g) in water (150 ml) in 1000 ml 3-necked flask. The resulting milky suspension was treated with zinc dust (5.7 g) in portions with rigorous stirring at such a rate as to keep the reaction mixture below 50° C. The addition required about 20 minutes, and after another two hours of stirring, the zinc oxide was removed by filtration and rinsed with hot water followed by methylene chloride (200 ml). The aqueous filtrate was once again extracted with methylene chloride (100 ml) and the combined methylene chloride extracts were washed with brine, dried over anhydrous sulphate, and directly taken for the next step.

Finally, the amidic nitrone product was obtained, as follows, using the following reaction:

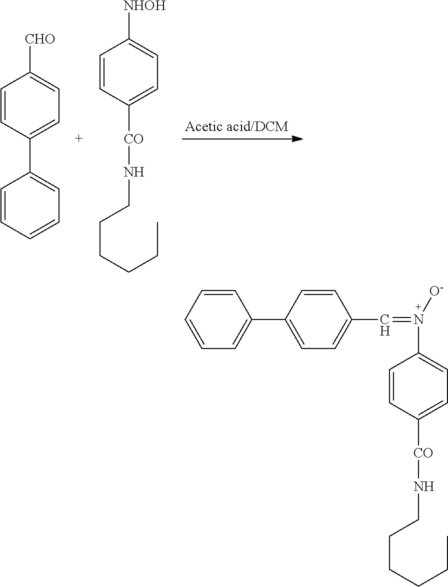

The compound N-(4-methylphenyl)-1-(4-phenylphenyl)methanimine oxide; N-hexylformamide was prepared as follows.

To a three-necked round-bottomed flask, equipped with reflux condenser, calcium chloride guard tube with nitrogen inlet and a magnetic stirrer, was charged with the methylene chloride extract (300 ml), biphenylaldehyde (5.46 g) and 100 ml of acetic acid. The mixture was stirred for 24 hrs at room temperature.

Next, the N-(4-methylphenyl)-1-(4-phenylphenyl)methanimine oxide; N-hexylformamide was prepared using the following reaction

The precipitated product was filtered and washed with water and ethanol to neutral. The product was dispersed in 200 ml of dichloromethane and stirred for 2 hr at 40° C. and filtered. The precipitate (product) was dried under vacuum. Yield: 10 g (80% with respect to aldehyde); HPLC Purity (% A): 92.0%.

### Example 4

This example illustrates the preparation of N-[4-(phenylcarbamoyl)phenyl]-1-(4-phenylphenyl)methanimine oxide.

In a first step, N-phenyl-4-nitrobenzamide was prepared, as follows, using the following reaction:

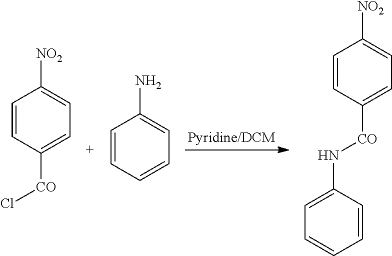

To a three-necked round-bottomed flask, equipped with reflux condenser, calcium chloride guard tube with nitrogen inlet and a magnetic stirrer, was charged dichloromethane (200 g), 4-nitro-benzoyl chloride (18.5 g, 0.1 mole) and pyridine (7.9 g, 0.1 mole). The mixture was cooled to 10-15° C. and aniline (9.5 g) added in lots by keeping the temperature below 30° C. The reaction mixture was stirred at room temperature (˜25° C.) for 2 hr. Then 5% HCl solution (300 ml) was added to the reaction mixture and stirred for 30 minutes. The aqueous layer was separated out and a further 300 ml of 5% sodium hydroxide washing was given to the reaction mixture. The aqueous layer was separated out and washed with water until neutral. After this, 200 ml of hexane was added to the reaction mixture, precipitating out the product completely. The thus obtained solid product was filtered and washed with water. The solid product was dried in an oven at 70° C. Yield: 22 g (90%); HPLC Purity (% A): 99.0%; Mass: m/z: 243.

In a second step, N-phenyl-4-hydroxylamine benzamide was prepared, as follows, using the following reaction:

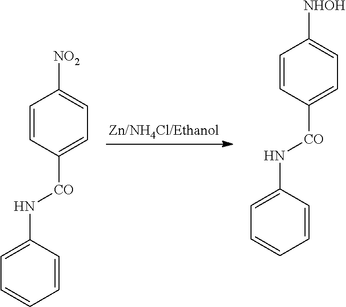

A solution of N-phenyl-4-nitrobenzamide (6 g) in warm 95% ethanol (100 ml) was mixed with ammonium chloride (1.43 g) in water (100 ml) in a 250-ml 3-necked flask. The resulting milky suspension was treated with zinc dust (3.5 g) in portions with rigorous stirring at such a rate as to keep the reaction mixture below 80° C. The addition required about 20 minutes, and after another two hours of stirring, the zinc oxide was removed by filtration and rinsed with hot water followed by methylene chloride (200 ml). The aqueous filtrate was once again extracted with methylene chloride (100 ml), and the combined methylene chloride extracts were washed with brine, dried over anhydrous sulphate, and directly taken for the next step.

In a third step, the N-[4-(phenylcarbamoyl)phenyl]-1-(4-phenylphenyl)methanimine oxide was prepared, as follows, using the following reaction:

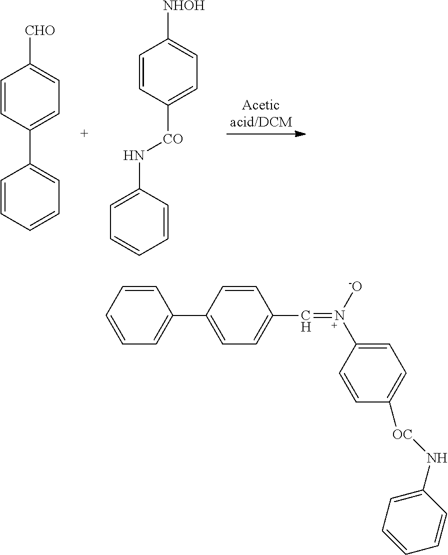

To a three-necked round-bottomed flask, equipped with reflux condenser, calcium chloride guard tube with nitrogen inlet and a magnetic stirrer, was charged methylene chloride extract (300 ml), biphenylaldehyde (2.6 g) and 100 ml of acetic acid. The mixture was stirred for 24 hrs at room temperature. The precipitated product was filtered and washed with water and ethanol to neutral, then dried under vacuum.

Yield: 4.5 g (76% with respect to aldehyde); HPLC Purity (% A): 90.0%; Mass: m/z: 408.

Based on the above methods of preparation, the dinitrone compounds in Table 4 and mononitrones in Table 5 were obtained.

TABLE 4

| | | Abs. | Abs. λ | | DSC | M⁺ |
|---|---|---|---|---|---|---|
| | | λ | cut | HPLC | Dec. | Mass |
| | | Max | off | Purity | Temp. | Spec- |
| No. | Structure | nm | nm | % | ° C. | trum |
| 1 | 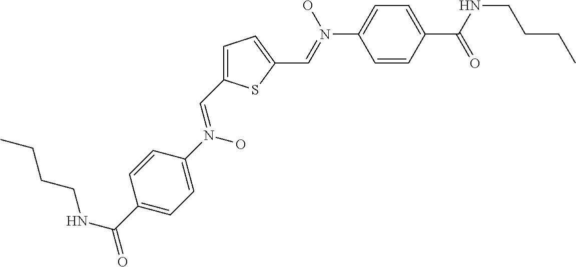 | 443 | 535 | 88 | 286° C. | 521.2 |
| 2 | 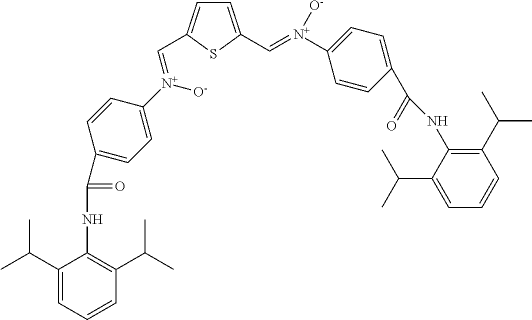 [SMILES: CC(C)c1cccc(C(C)C)c1NC(=O)c1ccc(/[N+]([O-])=C/c2ccc(/C=[N+](\[O-])c3ccc(C(=O)Nc4c(C(C)C)cccc4C(C)C)cc3)s2)cc1] | 440 | 534 | 90 | 314° C. | 729.5 |
| Comp. 3 | 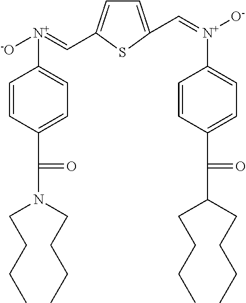 | 434 | 524 | 93 | 207° C. | 633.7 |
| 4 | 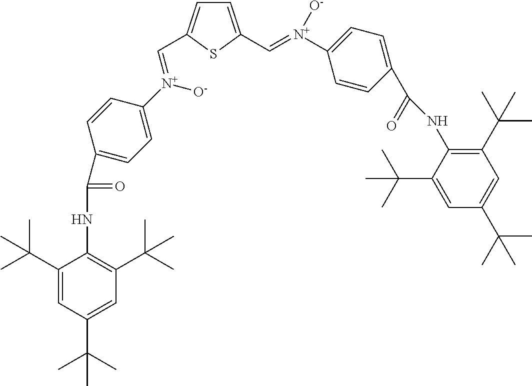 [SMILES: CC(C)(C)c1cc(C(C)(C)C)c(NC(=O)c2ccc(/[N+]([O-])=C/c3ccc(/C=[N+](\[O-])c4ccc(C(=O)Nc5c(C(C)(C)C)cc(C(C)(C)C)cc5C(C)(C)C)cc4)s3)cc2)c(C(C)(C)C)c1] | 439 | 534 | 89 | 282° C. | 643.5 |
| 5 | 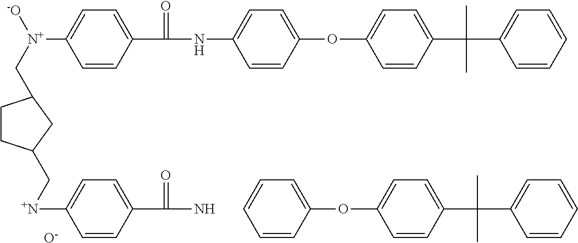 | 443 | 538 | 90 | 269° C. | 1003.0 |
| | 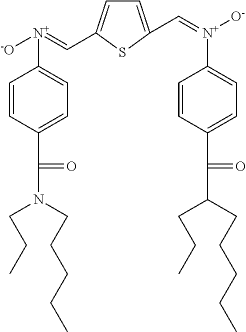 | | | | | |
| Comp. 6 | 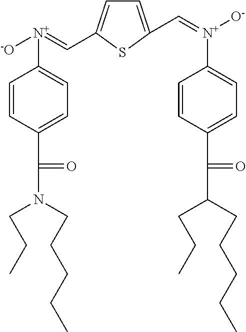 | 435 | 532 | 92 | 205° C. | 633.3 |

TABLE 5

| | | Abs. λ | Abs. Λ | HPLC | DSC | M⁺ |
|---|---|---|---|---|---|---|
| | | Max | cut off | Purity | (Dec. Temp. | Mass |
| No. | Structure | nm | nm | % | ° C.) | Spectrum |
| 7 | 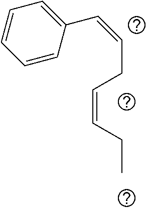 | 321 | 380 | 90 | 270° C. | 317.16 |
| 8 | 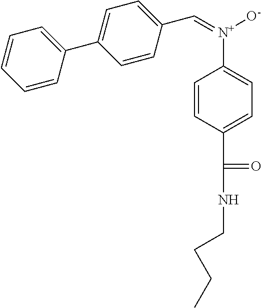 [SMILES: CCCCNC(=O)c1ccc(/[N+]([O-])=C\c2ccc(-c3ccccc3)cc2)cc1] | 343 | 409 | 90 | 263° C. | 401.5 |
| 9 | 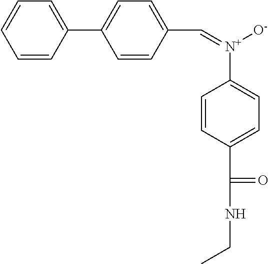 [SMILES: CCNC(=O)c1ccc(/[N+]([O-])=C\c2ccc(-c3ccccc3)cc2)cc1] | 342 | 408 | 90 | 263° C. | 373.12 |
| 10 | 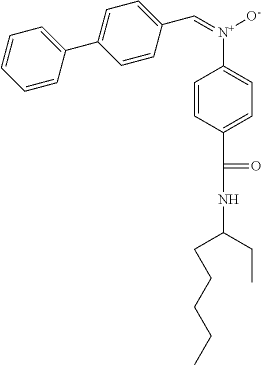 [SMILES: CCCCCC(CC)NC(=O)c1ccc(/[N+]([O-])=C\c2ccc(-c3ccccc3)cc2)cc1] | 342 | 408 | 93 | 253° C. | 429.5 |
| 11 | 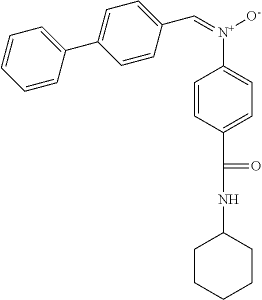 [SMILES: O=C(NC1CCCCC1)c1ccc(/[N+]([O-])=C\c2ccc(-c3ccccc3)cc2)cc1] | 344 | 410 | 90 | 287° C. | 399.2 |
| 12 | 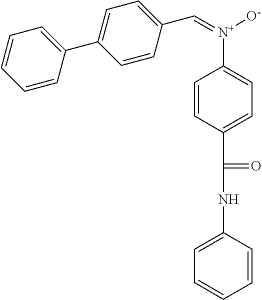 [SMILES: O=C(Nc1ccccc1)c1ccc(/[N+]([O-])=C\c2ccc(-c3ccccc3)cc2)cc1] | 344 | 411 | 90 | 291° C. | 393.15 |

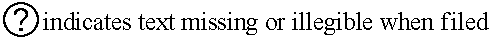

### Results

The physical properties, like melting point and decomposition temperature, of the above six di-nitrones and six mono-nitrones were studied using DSC. The mono-nitrones Compounds No. 11 and 12 showed the highest thermal stability of 291° C. This is believed to be due to Compounds No. 11 and 12 being more rigid and having lesser degrees of freedom, hence showing better packing in crystal.

Along with high heat stability, however, solubility was also an important parameter for the intended uses. Therefore, solubility of the synthesized amidic nitrones was studied in dichloromethane. It was found that the solubility of amidic nitrones prepared from the primary amine was less than 0.5% in dichloromethane, and those prepared from secondary amines had more than 10% solubility (Compound Nos. 3 and 4) in dichloromethane. Cancellation of the intermolecular hydrogen bonding via N,N-dialkylamine of nitrone tends to yield more soluble products but also less heat stable products.

Each of the amidic nitrones in Table 4 and 5, with different loadings, were then physically mixed with different polycarbonate grades and then processed between 260° C. and 280° C. The extruded pellets were subjected to HPLC analysis for measuring survival of the dye on heat treatment. HPLC results proved that the nitrones with amidic groups showed higher survival rates than compounds in the prior art.

Initially, three mono-nitrones (Compounds Nos. 8, 10, and 12) were taken for the extrusion trials. The processing temperature was 260° C.-280° C. The thermal stability of these nitrones was found to vary from 253-291° C., based on the DSC graph. Both Compound Nos. 8 and 10 were found to melt before their decomposition, whereas Example No. 12 was found to decompose directly without melting.

The extruded pellets obtained after processing of these three nitrones was transparent. This was an early indication of dyes being dissolved in low T_{g} polycarbonate at higher temperature. The extruded pellets were then submitted for quantification experiments. Based on the quantification of amidic mononitrones in extruded pellets, as quantified by normal phase HPLC methods using external calibration, the nitrones survival in extruded pellets was confirmed.

HPLC analyses of amidic nitrone were done using a silica column as the stationary phase and chloroform as the eluent. The polycarbonate being a macromolecule compared to amidic nitrone, the polymer did not have much interaction with the stationary phase and eluted very fast with a retention time of 2.5 min. The amidic nitrone, having both nitrone as well as a carbamide group, interacted with the stationary phase and mobile phase, so appeared at the retention time of 14.6 min The peak purity of the compound (retention time 14.6 min) was above 99%.

The Compound 12 mono-nitrone, after extrusion in the pellets, was quantified by an external calibration method. The calibration was done using the pure compound as standard (90% purity). A linear calibration curve with regression co-efficient above 99.999% was obtained in the range of 0.0005 mg/ml to 0.009 mg/ml.

Extruded pellets were weighed (200-300 mg) and dissolved in 15 ml chloroform by shaking 200 rpm at 45 min. The solution was directly injected into the HPLC. The quantity of amidic nitrone that survived was calculated by using the calibration curve. The results are given in Table 6, which provides a summary of nitrone survival after extrusion in the low Tg polycarbonate.

TABLE 6

| | | | % Survival after |
|---|---|---|---|
| No | Sample | Loading | extrusion at 270° C. |
| 1 | Compound 8 | 3% | 52 |
| 2 | Compound 10 | 3% | 50 |
| 3 | Compound 12 | 2% | 65 |

Table 6 shows that the percentage survival of the nitrone depends on its decomposition temperature. Compound 12 showed a maximum survival at 270° C. as compared to Compound Nos. 8 and 10, since Compound 12 has a more rigid compared to the other nitrones due to the strong intra-molecular hydrogen bonding. There is steric-hindrance between the molecules, since alkyl groups attached to the each nitrone can cause weakening of the intermolecular force of attraction, yielding a low decomposition temperature for the nitrone.

In view of the above, the amidic nitrones showed good thermal stability (270° C.-314° C.). These amidic nitrones were further used for extrusion and compression molding in low T_{g} polycarbonate at 260-280° C. The quantification results showed a survival of up to 65% for the dye, which would be useful in obtaining the desired high diffraction efficiency for holographic applications.

The holographic composition is advantageous in that it permits manufacture of a holographic storage medium in an efficient and cost effective manner, allowing for fast replication. Handling by the end-user is also facilitated.

The singular forms “a,” “an,” and “the” include plural referents unless the context clearly dictates otherwise.

The endpoints of all ranges directed to the same component or property are inclusive and independently combinable (e.g., ranges of “less than or equal to about 25 wt %, or, more specifically, about 5 wt % to about 20 wt %,” is inclusive of the endpoints and all intermediate values of the ranges of “about 5 wt % to about 25 wt %,” etc.).

The suffix “(s)” as used herein is intended to include both the singular and the plural of the term that it modifies, thereby including at least one of that term (e.g., the colorant(s) includes at least one colorants).

“Optional” or “optionally” means that the subsequently described event or circumstance can or can not occur, and that the description includes instances where the event occurs and instances where it does not.

Unless specifically defined otherwise, technical and scientific terms used herein have the same meaning as is commonly understood by one of skill in the art to which this invention belongs. Compounds are described using standard nomenclature. For example, any position not substituted by any indicated group is understood to have its valency filled by a bond as indicated, or a hydrogen atom. A dash (“-”) that is not between two letters or symbols is used to indicate a point of attachment for a substituent. For example, —CHO is attached through carbon of the carbonyl group.

The term “aromatic radical” refers to an array of atoms having a valence of at least one and comprising at least one aromatic group, i.e., a cyclic having 4n+2 “delocalized” electrons where “n” is an integer equal to 1 or greater. Aromatic radicals can include heteroatoms such as nitrogen, sulfur, selenium, silicon, and oxygen, or can be composed exclusively of carbon and hydrogen. The term “a C₃-C₁₀ aromatic radical” includes aromatic radicals having at least three but no more than 10 carbon atoms. The aromatic radical 1-imidazolyl (C₃H₂N₂—) represents a C₃ aromatic radical. Exemplary aromatic radicals include phenyl, pyridyl, furanyl, thienyl, naphthyl, phenylene, and biphenyl radicals. The aromatic radicals as that term is defined herein includes unsubstituted and substituted aromatic groups, and thus also include nonaromatic components. For example, a benzyl group is an aromatic radical, which comprises a phenyl ring (the aromatic group) and a methylene group (the nonaromatic component). The benzyl radical (C₇H₇—) represents a C₇ aromatic radical. Similarly a tetrahydronaphthyl radical is an aromatic radical comprising an aromatic group (C₆H₃) fused to a nonaromatic component —(CH₂)₄—. Substituents for the aromatic radicals include halogens, alkyl groups, alkenyl groups, alkynyl groups, haloalkyl groups, haloaromatic groups, conjugated dienyl groups, alcohol groups, ether groups, aldehyde groups, ketone groups, carboxylic acid groups, acyl groups (for example carboxylic acid derivatives such as esters and amides), amine groups, nitro groups, and the like.

Exemplary substituted aromatic radicals include halogenated aromatic radicals such as 4-trifluoromethylphenyl, hexafluoroisopropylidenebis(4-phen-1-yloxy) (—OPhC(CF₃)₂PhO—), 4-chloromethylphen-1-yl, 3-trifluorovinyl-2-thienyl, 3-trichloromethylphen-1-yl (3-CCl₃Ph-), 4-(3-bromoprop-1-yl)phen-1-yl (4-BrCH₂CH₂CH₂Ph-), and the like; and 4-allyloxyphen-1-oxy, 4-aminophen-1-yl (4-H₂NPh-), 3-aminocarbonylphen-1-yl (NH₂COPh-), 4-benzoylphen-1-yl, dicyanomethylidenebis(4-phen-1-yloxy) (—OPhC(CN)₂PhO—), 3-methylphen-1-yl, methylenebis(4-phen-1-yloxy) (—OPhCH₂PhO—), 2-ethylphen-1-yl, phenylethenyl, 3-formyl-2-thienyl, 2-hexyl-5-furanyl, hexamethylene-1,6-bis(4-phen-1-yloxy) (—OPh(CH₂)₆PhO—), 4-hydroxymethylphen-1-yl (4-HOCH₂Ph-), 4-mercaptomethylphen-1-yl (4-HSCH₂Ph-), 4-methylthiophen-1-yl (4-CH₃SPh-), 3-methoxyphen-1-yl, 2-methoxycarbonylphen-1-yloxy (methyl salicyl), 2-nitromethylphen-1-yl (2-NO₂CH₂Ph), 3-trimethylsilylphen-1-yl, 4-t-butyldimethylsilylphenl-1-yl, 4-vinylphen-1-yl, vinylidenebis(phenyl), and the like.

The term “cycloaliphatic radical” refers to a radical having a valence of at least one, and comprising an array of atoms that is cyclic but not aromatic. As defined herein, cycloaliphatic radicals do not contain an aromatic group, but can include one or more noncyclic components. For example, a cyclohexylmethyl group (C₆H₁₁CH₂—) is a cycloaliphatic radical that includes a cyclohexyl ring (the array of atoms which is cyclic but which is not aromatic) and a methylene group (the noncyclic component). The cycloaliphatic radicals can include heteroatoms such as nitrogen, sulfur, selenium, silicon and oxygen, or can be composed exclusively of carbon and hydrogen. The term “a C₃-C₁₀ cycloaliphatic radical” includes cycloaliphatic radicals having at least three but no more than 10 carbon atoms. The cycloaliphatic radical 2-tetrahydrofuranyl (C₄H₇O—) represents a C₄ cycloaliphatic radical. The cyclohexylmethyl radical (C₆H₁₁CH₂—) represents a C₇ cycloaliphatic radical. The aromatic radicals as that term is defined herein includes unsubstituted and substituted cycloaliphatic groups. Substituents include halogen groups, alkyl groups, alkenyl groups, alkynyl groups, haloalkyl groups, conjugated dienyl groups, alcohol groups, ether groups, aldehyde groups, ketone groups, carboxylic acid groups, acyl groups (for example carboxylic acid derivatives such as esters and amides), amine groups, nitro groups, and the like. Exemplary cycloaliphatic radicals include those comprising one or more halogen atoms, such as 2-trifluoromethylcyclohex-1-yl, 4-bromodifluoromethylcyclooct-1-yl, 2-chlorodifluoromethylcyclohex-1-yl, hexafluoroisopropylidene-2,2-bis(cyclohex-4-yl) (—C₆H₁₀C(CF₃)₂C₆H₁₀—), 2-chloromethylcyclohex-1-yl, 3-difluoromethylenecyclohex-1-yl, 4-trichloromethylcyclohex-1-yloxy, 4-bromodichloromethylcyclohex-1-ylthio, 2-bromoethylcyclopent-1-yl, 2-bromopropylcyclohex-1-yloxy (CH₃CHBrCH₂C₆H₁₀O—), and the like; and cycloaliphatic radicals include 4-allyloxycyclohex-1-yl, 4-aminocyclohex-1-yl (H₂C₆H₁₀—), 4-aminocarbonylcyclopent-1-yl (NH₂COC₅H₈—), 4-acetyloxycyclohex-1-yl, 2,2-dicyanoisopropylidenebis(cyclohex-4-yloxy) (—OC₆H₁₀C(CN)₂C₆H₁₀O—), 3-methylcyclohex-1-yl, methylenebis(cyclohex-4-yloxy) (—OC₆H₁₀CH₂C₆H₁₀O—), 1-ethylcyclobut-1-yl, cyclopropylethenyl, 3-formyl-2-terahydrofuranyl, 2-hexyl-5-tetrahydrofuranyl, hexamethylene-1,6-bis(cyclohex-4-yloxy) (—OC₆H₁₀(CH₂)₆C₆H₁₀O—), 4-hydroxymethylcyclohex-1-yl (4-HOCH₂C₆H₁₀—), 4-mercaptomethylcyclohex-1-yl (4-HSCH₂C₆H₁₀—), 4-methylthiocyclohex-1-yl (4-CH₃SC₆H₁₀—), 4-methoxycyclohex-1-yl, 2-methoxycarbonylcyclohex-1-yloxy (2-CH₃OCOC₆H₁₀O—), 4-nitromethylcyclohex-1-yl (NO₂CH₂C₆H₁₀—), 3-trimethylsilylcyclohex-1-yl, 2-t-butyldimethylsilylcyclopent-1-yl, 4-trimethoxysilylethylcyclohex-1-yl ((CH₃O)₃SiCH₂CH₂C₆H₁₀—), 4-vinylcyclohexen-1-yl, vinylidenebis(cyclohexyl), and the like.

The term “aliphatic radical” refers to an organic radical having a valence of at least one, a linear or branched array of atoms that is not cyclic, and at least one carbon atom. The aliphatic radicals can include heteroatoms such as nitrogen, sulfur, silicon, selenium, and oxygen or can be composed exclusively of carbon and hydrogen. A C₁-C₁₀ aliphatic radical contains at least one but no more than 10 carbon atoms. A methoxy group (—OCH₃) is an example of a C₁ aliphatic radical. An aliphatic radical, as that term is defined herein, includes unsubstituted and substituted aliphatic groups. Substitutents include halogen groups, alkyl groups, alkenyl groups, alkynyl groups, haloalkyl groups, conjugated dienyl groups, alcohol groups, ether groups, aldehyde groups, ketone groups, carboxylic acid groups, acyl groups (for example carboxylic acid derivatives such as esters and amides), amine groups, nitro groups, and the like. Exemplary substituted aliphatic radicals include halogenated aliphatic radicals such as trifluoromethyl, bromodifluoromethyl, chlorodifluoromethyl, hexafluoroisopropylidene, chloromethyl, difluorovinylidene, trichloromethyl, bromodichloromethyl, bromoethyl, 2-bromotrimethylene (—CH₂CHBrCH₂—), and the like; and allyl, aminocarbonyl (—CONH₂), carbonyl, 2,2-dicyanoisopropylidene (—CH₂C(CN)₂CH₂—), methyl (—CH₃), methylene (—CH₂—), ethyl, ethylene, formyl (i.e., —CHO), hexyl, hexamethylene, hydroxymethyl (i.e., —CH₂OH), mercaptomethyl (—CH₂SH), methylthio (—SCH₃), methylthiomethyl (—CH₂SCH₃), methoxy, methoxycarbonyl (CH₃OCO—), nitromethyl (—CH₂NO₂), thiocarbonyl, trimethylsilyl ((CH₃)₃Si—), t-butyldimethylsilyl, 3-trimethyoxysilylpropyl ((CH₃O)₃SiCH₂CH₂CH₂—), vinyl, vinylidene, and the like.

“Halogens” include fluorine, chlorine, bromine, and iodine.

The term “hydrocarbyl” refers broadly to a substituent comprising carbon and hydrogen, optional with at least one heteroatoms, for example, oxygen, nitrogen, halogen, or sulfur; “alkyl” refers to a straight or branched chain monovalent hydrocarbon group; “alkylene” refers to a straight or branched chain divalent hydrocarbon group; “alkylidene” refers to a straight or branched chain divalent hydrocarbon group, with both valences on a single common carbon atom; “alkenyl” refers to a straight or branched chain monovalent hydrocarbon group having at least two carbons joined by a carbon-carbon double bond; “cycloalkyl” refers to a non-aromatic monovalent monocyclic or multicylic hydrocarbon group having at least three carbon atoms, “cycloalkenyl” refers to a non-aromatic cyclic divalent hydrocarbon group having at least three carbon atoms, with at least one degree of unsaturation; “aryl” refers to an aromatic monovalent group containing only carbon in the aromatic ring or rings; “arylene” refers to an aromatic divalent group containing only carbon in the aromatic ring or rings; “alkylaryl” refers to an aryl group that has been substituted with an alkyl group as defined above, with 4-methylphenyl being an exemplary alkylaryl group; “arylalkyl” refers to an alkyl group that has been substituted with an aryl group as defined above, with benzyl being an exemplary arylalkyl group; “acyl” refers to an alkyl group as defined above with the indicated number of carbon atoms attached through a carbonyl carbon bridge (—C(═O)—); “alkoxy” refers to an alkyl group as defined above with the indicated number of carbon atoms attached through an oxygen bridge (—O—); and “aryloxy” refers to an aryl group as defined above with the indicated number of carbon atoms attached through an oxygen bridge (—O—).

Unless otherwise indicated, each of the foregoing groups can be unsubstituted or substituted, provided that the substitution does not significantly adversely affect synthesis, stability, or use of the compound. The term “substituted” as used herein means that at least one hydrogen on the designated atom or group is replaced with another group, provided that the designated atom's normal valence is not exceeded. When the substituent is oxo (i.e., ═O), then two hydrogens on the atom are replaced. Combinations of substituents and/or variables are permissible provided that the substitutions do not significantly adversely affect synthesis or use of the compound.

Exemplary groups that can be present on a “substituted” position include, but are not limited to, halogen; cyano; hydroxyl; nitro; azido; alkanoyl (such as a C2-C6 alkanoyl group such as acyl or the like); carboxamido; alkyl groups (typically having 1 to about 8 carbon atoms, or 1 to about 6 carbon atoms); cycloalkyl groups, alkenyl and alkynyl groups (including groups having at least one unsaturated linkages and from 2 to about 8, or 2 to about 6 carbon atoms); alkoxy groups having at least one oxygen linkages and from 1 to about 8, or from 1 to about 6 carbon atoms; aryloxy such as phenoxy; alkylthio groups including those having at least one thioether linkages and from 1 to about 8 carbon atoms, or from 1 to about 6 carbon atoms; alkylsulfinyl groups including those having at least one sulfinyl linkages and from 1 to about 8 carbon atoms, or from 1 to about 6 carbon atoms; alkylsulfonyl groups including those having at least one sulfonyl linkages and from 1 to about 8 carbon atoms, or from 1 to about 6 carbon atoms; aminoalkyl groups including groups having at least one N atoms and from 1 to about 8, or from 1 to about 6 carbon atoms; aryl having 6 or more carbons and at least one rings, (e.g., phenyl, biphenyl, naphthyl, or the like, each ring either substituted or unsubstituted aromatic); arylalkyl having 1 to 3 separate or fused rings and from 6 to about 18 ring carbon atoms, with benzyl being an exemplary arylalkyl group; or arylalkoxy having 1 to 3 separate or fused rings and from 6 to about 18 ring carbon atoms, with benzyloxy being an exemplary arylalkoxy group.

The term “optically transparent” as applied to an optically transparent substrate or an optically transparent plastic material means that the substrate or plastic material has an absorbance of less than 1. That is, at least 10 percent of incident light is transmitted through the material at least one wavelength in a range between about 300 nanometers and about 1500 nanometers. For example, when configured as a film having a thickness suitable for use in holographic data storage said film exhibits an absorbance of less than 1 at least one wavelength in a range between about 300 nanometers and about 1500 nanometers.

The terms “photochemically reactive” and “photoreactive” have the same meaning and are interchangeable.

The term “volume element” means a three dimensional portion of a total volume.

The term “optically readable datum” is a datum that is stored as a hologram patterned within one or more volume elements of an optically transparent substrate.

All cited patents, patent applications, and other references are incorporated herein by reference in their entirety. However, if a term in the present application contradicts or conflicts with a term in the incorporated reference, the term from the present application takes precedence over the conflicting term from the incorporated reference.

While the invention has been described with reference to exemplary embodiments, it will be understood by those skilled in the art that various changes can be made and equivalents can be substituted for elements thereof without departing from the scope of the invention. In addition, many modifications can be made to adapt a particular situation or material to the teachings of the invention without departing from the essential scope thereof. Therefore, it is intended that the invention not be limited to the particular embodiment disclosed as the best mode contemplated for carrying out this invention.

1. A holographic storage medium comprising a polymer binder and a nitrone compound according to the formula: 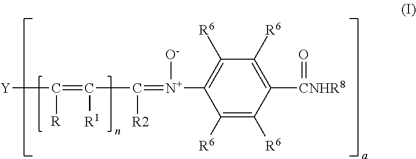
wherein Y is a monovalent or multivalent C₂-C₃₀ organic radical; each of R, R¹, and R² is independently hydrogen, an C₁-C₈ aliphatic, or a C₆-C₁₃ aromatic radical; each R⁶ is independently hydrogen, halo, cyano, nitro, a C₁-C₈ aliphatic radical, or a C₆-C₁₃ aromatic radical; R⁸ is hydrogen, a C₁-C₈ aliphatic radical, a C₆-C₈ cycloalkyl radical, or a C₆-C₁₃ aromatic radical; a is an integer of 1 to 4; and n is an integer of 0 to 4.

2. The holographic storage medium of claim 1, wherein at least two R⁶ groups are hydrogen.

3. The holographic storage medium of claim 1, wherein a is an integer of 2 to 4, and Y is a multivalent organic radical that is a C₁-C₂₀ aliphatic radical, a C₃-C₂₀ cycloaliphatic radical, or a C₃-C₃₀ aromatic radical.

4. The holographic storage medium of claim 3, wherein Y is a divalent organic radical and a is 2.

5. The holographic storage medium of claim 4, wherein Y is a C₃-C₃₀ aromatic radical.

6. The holographic storage medium of claim 5, wherein Y is phenylene or a divalent radical derived from thiophene.

7. The holographic storage medium of claim 1 wherein when a is 1 and Y is a monovalent radical according to the formula (R³)_{b}-Q-R⁴— or R⁵—, wherein Q is a monovalent, divalent, or trivalent C₁-C₆ organic linking group; R³ is a C₁-C₈ aliphatic radical or a C₆-C₁₃ aromatic radical; b is an integer of 0 to 3; R⁴ is a C₆-C₁₃ aromatic radical; R⁵ is a C₆-C₂₀ aromatic radical.

8. The holographic storage medium of claim 7 wherein n is zero, R² is hydrogen, and R⁴ and R⁵ is phenyl.

9. The holographic storage medium of claim 1 wherein n is zero, R² is hydrogen, and R⁴ and R⁵ is phenyl.

10. The holographic storage medium of claim 1, wherein R⁸ is a C₁-C₈ aliphatic group.

11. The holographic storage medium of claim 1 wherein the R⁸ group is a C₁-C₆ alkyl, a phenyl, or a cyclohexyl group.

12. The holographic storage medium of claim 1 wherein the R⁸ group is a phenyl group.

13. The holographic storage medium of claim 1 wherein the nitrone compound is according to the formula: 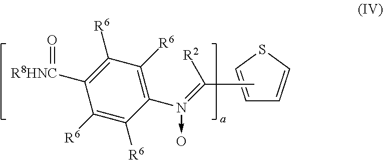
wherein a is an integer from 2 to 4; and R², R⁶, and R⁸ are as defined in claim 1.

14. The holographic storage medium of claim 13, wherein a is 2.

15. The holographic storage medium of claim 14 wherein R² and R⁶ are each hydrogen and R⁸ is an alkyl, cyclohexyl, or phenyl group.

16. The holographic storage medium of claim 15 wherein the phenyl group is substituted with 1-3 C₁-C₆ alkyl groups.

17. The holographic storage medium of claim 1, wherein the nitrone compound is selected from the group consisting of

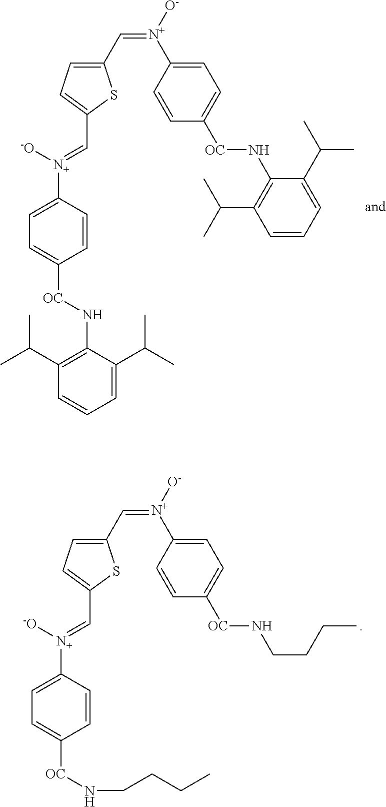

18. The holographic storage medium of claim 1, wherein the polymer binder is a thermoplastic.

19. The holographic storage medium of claim 1, wherein the polymer binder is a polycarbonate, a polyestercarbonate, or a mixture thereof.

20. The holographic storage medium of claim 1, wherein the polymer binder has a glass transition temperature of at least 120° C.

21. A compound according to the formula: 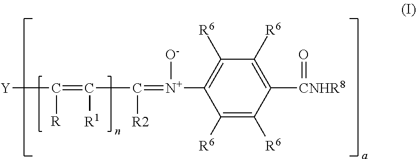
wherein Y is a monovalent or multivalent C₂-C₃₀ organic radical; each of R, R¹, and R² is independently hydrogen, an C₁-C₈ aliphatic, or a C₆-C₁₃ aromatic radical; each R⁶ is independently hydrogen, halo, cyano, nitro, a C₁-C₈ aliphatic radical, or a C₆-C₁₃ aromatic radical; R⁸ is hydrogen, a C₁-C₈ aliphatic radical, a C₆-C₈ cycloalkyl radical, or a C₆-C₁₃ aromatic radical; a is an integer of 1 to 4; and n is an integer of 0 to 4.

22. The compound of claim 21, wherein at least two R⁶ groups are hydrogen.

23. The compound of claim 21, wherein Y is a multivalent organic radical that is a C₁-C₂₀ aliphatic radical, a C₃-C₂₀ cycloaliphatic radical, or a C₃-C₃₀ aromatic radical.

24. The compound of claim 21, wherein Y is a divalent organic radical and a is 2.

25. The compound of claim 24, wherein Y is a C₃-C₃₀ aromatic radical.

26. The compound of claim 25, wherein Y is phenylene or a divalent radical derived from thiophene.

27. The compound of claim 26 wherein when a is 1 and Y is a monovalent radical according to the formula (R³)_{b}-Q-R⁴— or R⁵—, wherein Q is a monovalent, divalent, or trivalent C₁-C₆ organic linking group; R³ is a C₁-C₈ aliphatic radical or a C₆-C₁₃ aromatic radical; b is an integer of 0 to 3; R⁴ is a C₆-C₁₃ aromatic radical; R⁵ is a C₆-C₂₀ aromatic radical.

28. The compound of claim 27 wherein n is zero, R² is hydrogen, and R⁴ and R⁵ is phenyl.

29. The compound of claim 21 wherein n is zero, R² is hydrogen, and R⁴ and R⁵ is phenyl.

30. The compound of claim 21, wherein R⁸ is a C₁-C₈ aliphatic group.

31. The compound of claim 21 wherein the R⁸ group is a C₁-C₆ alkyl, a phenyl, or a cyclohexyl group.

32. The compound of claim 21 wherein the R⁸ group is a phenyl group.

33. The compound of claim 21 wherein the nitrone compound is according to the formula: 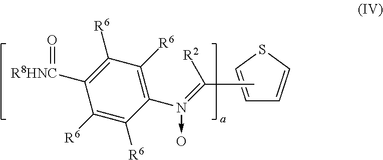
wherein a is an integer from 2 to 4; and R², R⁶, and R⁸ are as defined in claim 21.

34. The compound of claim 33, wherein a is 2.

35. The compound of claim 34 wherein R² and R⁶ are each hydrogen and R⁸ is an alkyl, cyclohexyl, or phenyl group.

36. The compound of claim 35 wherein the phenyl group is substituted with 1-3 C₁-C₆ alkyl groups.

37. The compound of claim 21 wherein the compound is selected from the group consisting of

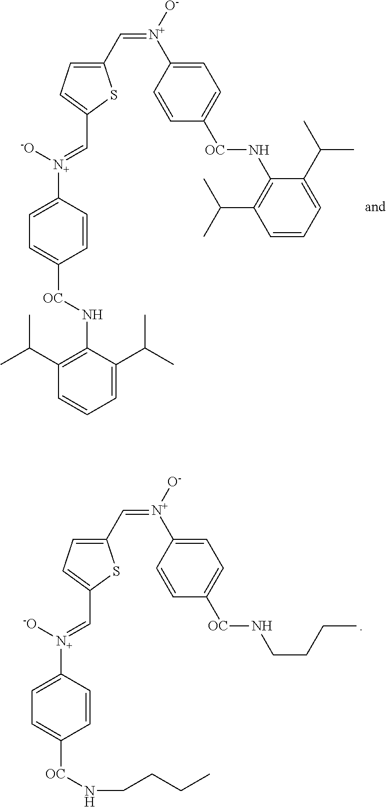

38. A method of manufacturing a holographic storage medium, comprising:
mixing a compound according to claim 21 with a molten thermoplastic polymer binder to form a mixture
forming the mixture into the holographic storage medium.

39. The method according to claim 38, wherein the molten thermoplastic polymer binder is at a temperature of at least 120° C.
